(19) United States
(12) Patent Application Publication
(10) Pub. No.: US 2004/0097833 A1
(43) Pub. Date: May 20, 2004

(54) Mammary fluid collection device and kit

(76) Inventors: Richard L. Mueller, JR., Byron, CA (US); James Kermode, Los Altos, CA (US); Tammy Gravelle, Palo Alto, CA (US); Todd Thompson, San Jose, CA (US); Douglas Murphy-Chutorian, Palo Alto, CA (US)

Correspondence Address: OLSON & HIERL, LTD., 36th Floor, 20 North Wacker Drive, Chicago, IL, 60606, US

(21) Appl. No.: 10/299,163

(22) Filed: Nov. 19, 2002

(51) Int. Cl. A61B 5/00

(52) U.S. Cl. 600/573

(57) ABSTRACT

The present invention provides a collection device and kit for collecting an exudate from a mammary duct that terminates at a human breast nipple. The collection device comprises a hollow receptacle defining an interior volume and having an open aperture sized to circumscribe the nipple and defined by a rim. The collection device further includes an absorbent pad in the interior volume, which pad is positionable to contact the nipple when the receptacle is situated over the nipple. In order to keep the collection device in place, a pressure sensitive adhesive is also provided on the collection device.

### TECHNICAL FIELD OF THE INVENTION

The invention relates to an apparatus and kit for collecting mammary fluid samples. More specifically, the invention relates to an apparatus and kit for obtaining mammary fluid samples from the mammary glands of a human for evaluating, diagnosing, and managing breast diseases, including infections, pre-cancerous conditions, cancer susceptibility and cancer. A method for using the present invention is also disclosed.

### BACKGROUND OF THE INVENTION

Breast cancer is one of the health threats most feared by American women, and is the most common form of cancer in women. A key to treatment is early detection. For example, annual mammograms is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. As such, more sensitive and reliable methods and devices are needed to detect cancerous, pre-cancerous, and other cancer indicators of the breast at an early stage. Such methods and devices could significantly improve breast cancer survival. While breast cancer is most common among women, in rare instances the human male may also have occurrences of breast cancer.

Other methods of detecting breast cancer are based on the fact that a vast majority of instances of breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary fluid typically contains cells and solid cellular debris or products that can also be used in cytological or immunological assays.

An early method based on this analysis of mammary fluid involves analysis of the mammary fluid that is excreted by the mammary ducts without outside intervention. In other words, if a mammary duct is discharging a fluid, this fluid is analyzed. This process, however, requires that the mammary ducts are actually excreting fluid. Often that is not the case even though breast cancer markers may be present. As such, need exists in the art for more widely applicable, methods and devices to obtain samples for use in evaluating, diagnosing and managing breast disease including cancer. Such methods and devices should be affordably available and be utilized at home so as to avoid the necessity of costly and time consuming doctor's visits.

One procedure based on the analysis of mammary fluid is called ductal lavage. This method entails introducing of saline via a small catheter into the mammary ducts, and removal of the introduced solution by vacuum from the mammary ducts for analysis. One problem with such a procedure is that it must be performed by a doctor, thus, home testing is not possible. As such, many women may not be tested until a scheduled appointment with a doctor. Another method that has been developed is shown in U.S. Pat. No. 6,328,709 to Hung et. al., which provides a device for aspirating and retrieving breast duct fluid through use of a vacuum to cause the breast nipple to exude the fluid in a manner similar to breast pumps utilized by lactating women to collect breast milk. Similar to ductal lavage, the method and device shown in U.S. Pat. No. 6,328,709 requires costly equipment and specialized knowledge. Accordingly, the method and device shown in U.S. Pat. No. 6,328,709 is typically performed by a doctor.

Also, in both examples, a vacuum or suction is used to draw mammary duct fluid from the mammary ducts. This often results in discomfort to the patient. Typically, both procedures must also be performed as part of a doctor's visit.

The present invention overcomes these shortcomings of the prior art by enabling a person to collect mammary duct exudate with a collection device that may be provided in a kit to be used preferably at home, similar to home pregnancy tests. The present invention also avoids the discomfort typically encountered due to the vacuuming techniques employed by the prior art in as much as mammary duct exudate can be collected without vacuum assist.

### SUMMARY OF THE INVENTION

A convenient, self-administrable collection device and kit for collecting an exudate from a mammary duct is provided. The collection device comprises a hollow receptacle defining an interior volume and having an open aperture defined by a rim and sized to circumscribe a human breast nipple. An absorbent pad is present in the interior volume. The absorbent pad is positionable to contact the nipple when the receptacle is situated over the nipple. The absorbent pad can be made of a relatively thin sheet of a white or lightly colored absorbent material that enhances visibility of moisture when present in or on the pad. Additionally, the absorbent pad may include a diagnostic composition and/or coloring that contrasts and highlights the various colors of relatively higher risk exudates that may be encountered, e.g., green, brown, yellow and red, and the like. The absorbent pad can also include a blood highlighting material that changes color, e.g., guaiac reagent that turns blue (Hemoccult® guaiac test), in the presence of red blood cells. In order to keep the collection device in place, a pressure sensitive adhesive is also provided, preferably on the rim.

Optionally, the collection device may also include a removable cover situated over the aperture to maintain sterility of the absorbent pad prior to use. This cover is designed to protect the hollow receptacle and the absorbent pad therein from contaminants prior to use as well as after use to preserve the collected sample. The cover may optionally be configured to remain attached to the collection device to prevent accidental loss, e.g., with a hinged attachment to the collection device, and the like.

In a preferred form, the rim of the collection device is a flange of either a flexible or a non-flexible material. The flange may be shaped to conform substantially to the breast or may be disc shaped as desired. The rim, preferably a flange or a ring, is provided with a pressure sensitive adhesive. Prior to use, the adhesive is preferably covered by a strip or sheet of release material. The pressure sensitive adhesive permits securement of the collecting device to the breast while leaving both hands free to massage the breast to express an aliquot of a mammary fluid discharge that may be present.

To position the absorbent pad in the receptacle, a reciprocating or telescoping mechanism may be provided to adjust the relative distance from the rim to the absorbent pad since nipple height can vary greatly among individuals. The reciprocating or telescoping mechanism may take any form suitable as long as the relative distance from the rim to the absorbent pad can be adjusted. For example, a two part telescoping receptacle may be formed having a hollow portion and a plunger portion, with the hollow portion and the plunger portion in a telescoping relationship relative to one another. In another embodiment, the receptacle may be connected to the rim by a hinge such that the hinge allows the receptacle and absorbent pad therein to be moved toward to the nipple after positioning. In yet another embodiment, the hollow section can be a flexible material provided with collapsible and extendable pleats. In a further embodiment, the receptacle may include two parts that are in a threaded relationship to each other such that the portion carrying the absorbent pad may be turned about an axis substantially normal to the rim so as to increase or decrease the relative distance between the absorbent pad and the rim, and thus the nipple, when the collection device is in place. In still a further embodiment, the absorbent pad can be carried on an adhesive strip and circumscribed by a ring that defines the rim of the hollow receptacle as well as the receptacle itself. In some embodiments the adhesive strip can extend beyond the periphery of the ring and serve to hold the collection device in place when in use. In the alternative, the ring can be provided with a pressure sensitive adhesive for the same purpose. A protective cover made from a sheet of release paper occludes the absorbent pad as well as the exposed adhesive surface or surfaces prior to use and after a mammary exudate sample has been collected.

The various embodiments of the present collection devices may also include a viewing window. This window may be situated at a proximal end of the receptacle, and can optionally include a magnifying lens that permits closer inspection of the absorbent pad after the mammary fluid sample has been collected. Depending on the appearance, consistency, and amount of mammary fluid collected, the user may wish to seek further analysis or a consultation with doctor.

Unlike the devices and methods of the prior art, the mammary duct fluid is not collected using a vacuum assist. Instead, the collection device is secured in place with the pressure sensitive adhesive with the absorbent pad positioned in contact with the nipple, and the breast is massaged to express any fluid that may be present. If desired, the breast may be warmed to enhance exudate flow by reason of capillary dilation in the mammary tissue and the opening of ducts and pores in the mammary glands. The present invention may also be embodied in a kit for home self-examinations.

In addition to the collecting device, the kit also includes an implement for exfoliating the nipple region to remove keratin particles that may be blocking mammary duct openings. This exfoliating implement may comprise a brush, textured pad, or other mildly abrasive device for removing dead skin cells or dried mammary fluid. The exfoliating implement may also be an astringent or other cleansing solution, e.g., a detergent, to remove anything that may inhibit extrusion of the mammary fluid. The kit further includes a sterile alcohol wipe for aseptically cleaning the nipple before collecting the mammary fluid sample. A dry wipe is also included in the kit to dry the cleansed region. Preferably the kit contains more than one collecting device so that both breasts may be examined substantially concurrently.

The present sample collection devices are well suited for periodic home use. High risk patients can readily self-administer mammary duct fluid collection at prescribed intervals between mammographic examinations. In use, the collection device is adhered to the breast such that the aperture circumscribes the nipple. The absorbent pad is then adjusted to contact the breast nipple. The breast is then massaged to physically stimulate the breast tissue so as to express any breast fluid from the mammary ducts for collection on the absorbent pad. Massaging is effected generally in the direction from the chest wall towards the areola and the nipple. Thereafter, the collection device is removed from the breast, and the collected fluid aliquot inspected or analyzed.

### BRIEF DESCRIPTION OF THE DRAWINGS

In the drawings,

FIG. 1 is a perspective view of a collection device embodying the present invention;

FIG. 2 is a bottom schematic view of the collection device shown in FIG. 1;

FIG. 3 is side elevational view of the collection device shown in FIG. 1 with a portion broken away to show interior detail;

FIG. 4 is a partially cut-away schematic perspective view of another collection device that embodies the present invention;

FIG. 5 is a sectional view of another collection device that embodies the present invention;

FIG. 6 is a plan view of yet another collection device that embodies the present invention;

FIG. 7 is a side-elevational view, partly in section, of the embodiment shown in FIG. 6;

FIG. 8 illustrates a kit embodying the present invention, and constituted by a collection device, an exfoliating implement, and wipes; and

FIG. 9 illustrates another kit embodying the present invention.

### DESCRIPTION OF THE PREFERRED EMBODIMENT OF THE INVENTION

The invention disclosed herein is, of course, susceptible of being embodied in many different manners. Shown in the drawings and described herein below in detail is a preferred embodiment of the invention. It is to be understood, however, that the present disclosure is an exemplification of the principles of the invention and does not limit the invention to the illustrated embodiments.

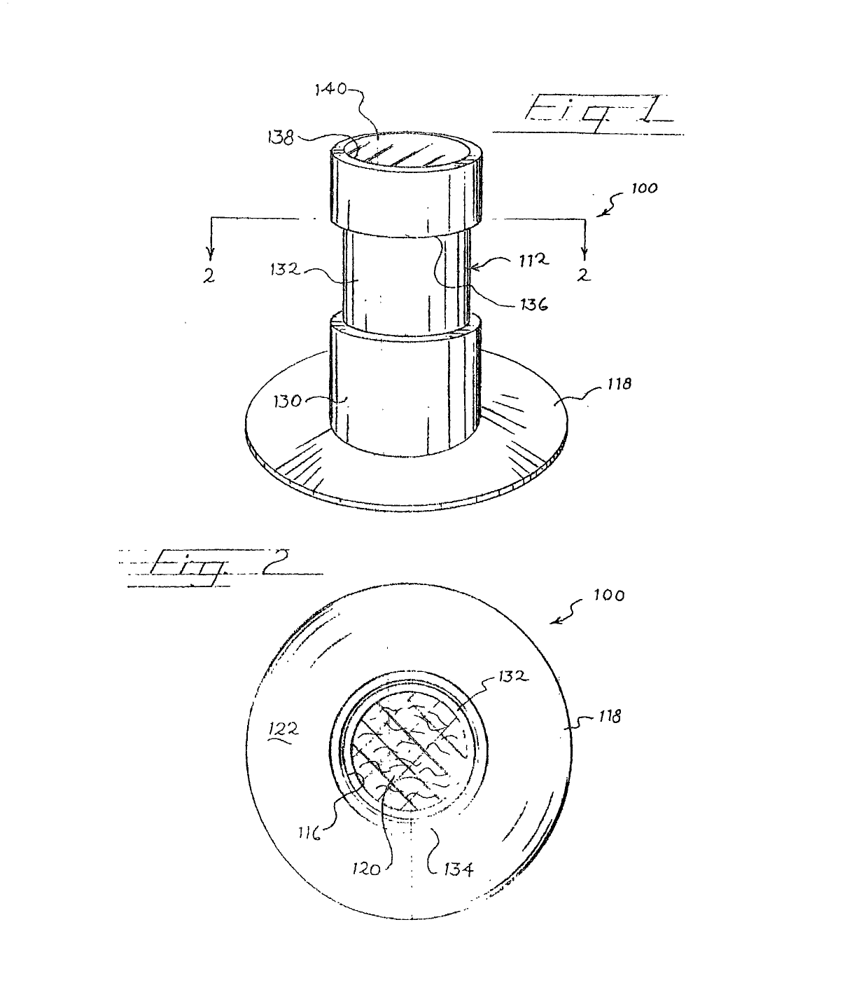

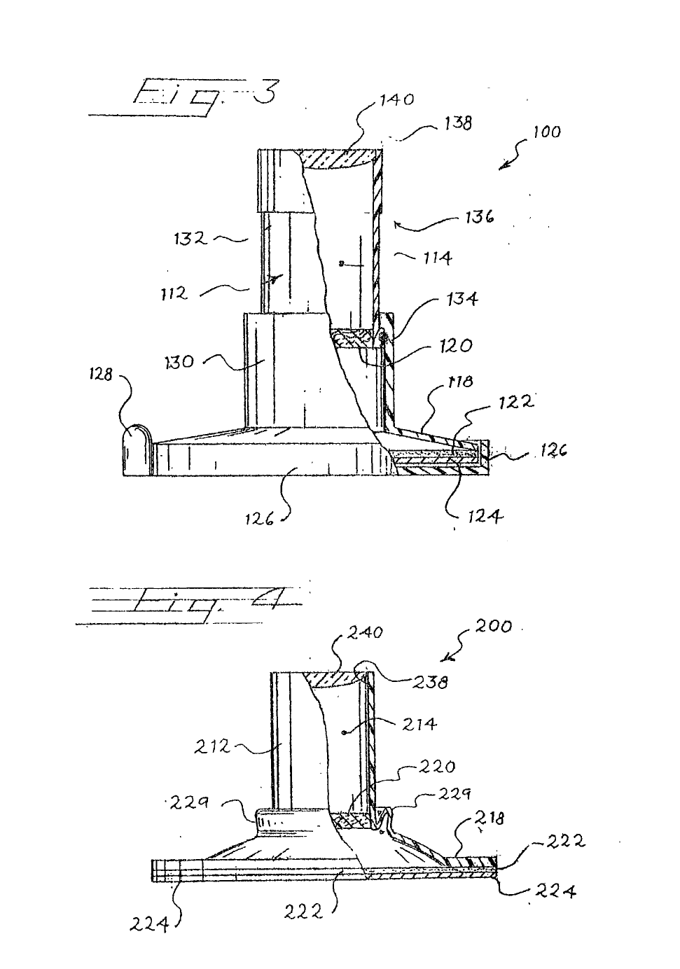

Referring to FIGS. 1-3, inclusive, collection device 100 comprises a hollow receptacle 112 having an open aperture 116 (FIG. 2) sized to circumscribe the nipple (not shown) and defined by a rim 118. Hollow receptacle 112 also defines an interior volume 114 (FIG. 3). The collection device 100 further includes an absorbent pad 120 in the interior volume 114, which is positionable to contact the nipple when the receptacle 112 is situated over the nipple. In order to keep the collection device 100 in place, a pressure sensitive adhesive 122 is also included on the rim 118. In a preferred form, the rim 118 of the collection device is a flange of either a flexible or a non-flexible material. The flange may be shaped to conform to the breast contour or may be disc shaped. Prior to use, the adhesive 122 is preferably covered by a strip or sheet of release material 124, e.g., a silicone coating.

An optional protective cover 126 is shown in FIG. 3. Protective cover 126 can be configured to protect absorbent pad 120 and receptacle 112 from contamination prior to use. Preferably the cover 126 fits over the rim 118. Protective cover 126 can optionally be secured to the collection device 100 by way of a hinge 128 or the like to prevent accidental loss or misplacement.

To contact the nipple, the absorbent pad 120 in receptacle 112 is positioned through use of two telescoping parts, a hollow tubular portion 130 and a plunger portion 132. A seal 134, such as an o-ring, is also preferred. The absorbent pad 120 is located at a distal end of the plunger portion 132. The hollow portion 130 and the plunger portion 132 of receptacle 112 are in a telescopically sliding engagement with one another. In this manner, the plunger portion 132 can slide relative to the hollow portion 130 in order to adjust the relative distance from the absorbent pad 120 to the rim 118, and thereby accommodate a variety of nipple heights. In FIG. 3, the plunger portion 132 is preferably cylindrical with a circumferential stop 136, such as a ridge, which is slightly larger in diameter than the inner circumference of the hollow portion 130. As such, the extent to which the absorbent pad 120 may be positioned relative to rim 118 is limited.

The collection device 100 may also include a viewing window 138. This window may be situated at a distal end of the receptacle 112, and can optionally include a magnifying lens 140 that allows closer inspection of the absorbent pad 120 after the sample of mammary fluid has been collected. Depending on the appearance, consistency, and amount of mammary fluid collected, the user may wish to seek further analysis, or at least consult with a physician.

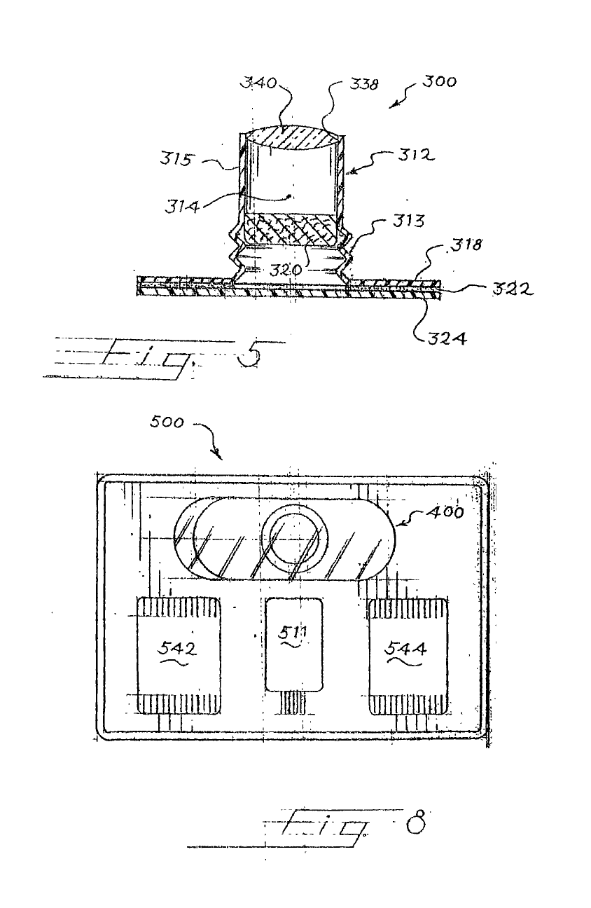

Collection device 200 shown in FIG. 4 comprises hollow receptacle 212 defining an interior volume 214 and having an open aperture sized to circumscribe a nipple. The open aperture is defined by rim 218. An absorbent pad 220 is positionably located in the interior volume 214 of the receptacle 212, and can be positioned to contact a breast nipple when the receptacle 212 is situated over the nipple. Similar to the previous embodiment, a pressure sensitive adhesive 222 is provided on the rim 218 and is preferably covered by a release strip 224 prior to use. In this embodiment, as well as the other embodiments, it is contemplated that a removable cover (not shown) is provided over the aperture, and preferably the entire rim 218.

In lieu of the reciprocating mechanism of the illustrated embodiment in FIGS. 1-3, the embodiment shown in FIG. 4 utilizes a hinge region 229 unitary with receptacle 212 and rim 218 to permit the receptacle 212 and absorbent pad 220 therein to be moved toward the nipple. The flexible hinge region 229 can be constructed of a resilient material such that the receptacle 212 can be raised and lowered relative to the rim 218. In a preferred form, the rim 218 of the collection device 220 is a flange, which may be either a flexible or a rigid material, and may be shaped to conform to the breast contour, or may be disc shaped as before. The collection device 200 may also include a viewing window 238. This window may be formed at the proximal end of the receptacle 212, and can optionally include a magnifying lens 240.

Yet another embodiment of the present invention is shown in FIG. 5. In this particular embodiment, the collection device 300 comprises a receptacle 312 formed of a flexible material having a pleated portion 313 and a smooth wall portion 315. The pleated wall portion 313 may be collapsed or extended as desired to adjust the location of the absorbent pad 320. Preferably absorbent pad 320 is located at the junction of pleated wall portion 313 and smooth wall portion 315. Pressure sensitive adhesive 322 on rim 318 is protected by release sheet 324. A viewing window 338 is provided at the proximal end of hollow receptacle 312 and is optionally equipped with magnifying lens 340.

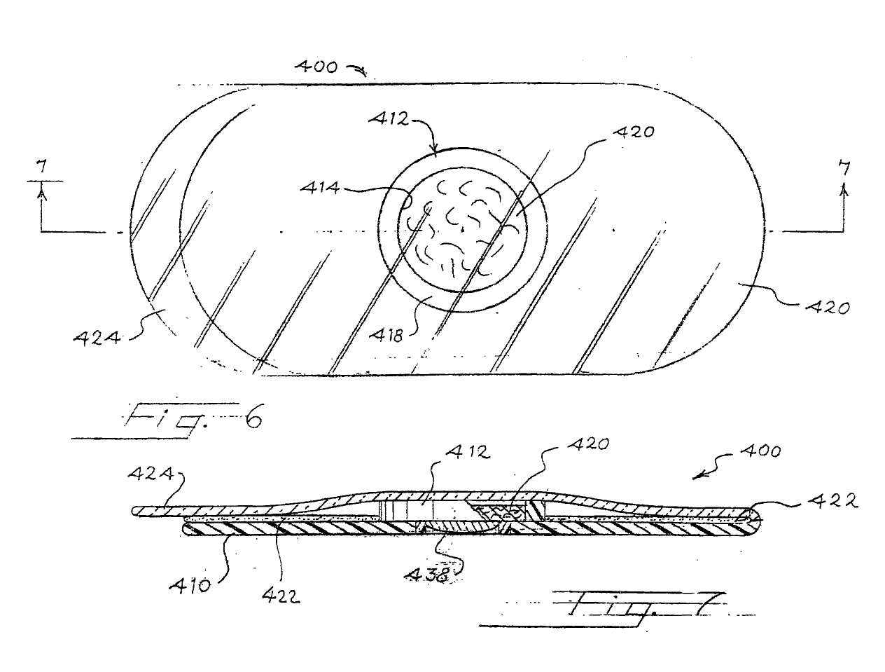

A further embodiment of the present invention is illustrated in FIGS. 6 and 7. Collection device 400 is provided with a hollow receptacle 412 defined by rim 418 in the form of a ring, which also defines an interior volume 414. The ring can be made of a radio-opaque material, if desired, or the absorbent pad can include radio-opaque markers, to facilitate identification of nipple locations during a mammographic examination in instances where the device of the present invention is utilized in conjunction with a mammographic examination. For example, to capture exudate resulting from mammographic clamping. Other orientation assisting markers such as radio-opaque dots or quadrant identifiers can be present as well.

Absorbent pad 420 is located within the periphery of the ring and in interior volume 414. A transparent release sheet 424 is positioned over pressure sensitive adhesive 422 provided on oblong base sheet 410 which also retains rim 418 and pad 420 in place. Base sheet 410 is shown as having an oblong configuration; however, the base sheet can have a wide variety of configurations such as circular, elliptical, rectangular, and the like. Base sheet 410 preferably is flexible so as to comply substantially with the contour of the breast. More preferably, the base sheet 410 is made of an elastomeric material, such as the backing for an adhesive bandage. A viewing window 438, with or without a magnifying lens, can be provided in base sheet 410, if desired. In the alternative, the transparent release sheet can include a magnifying lens, e.g., a Fresnel lens, over pad 420 to facilitate inspection of any collected exudate.

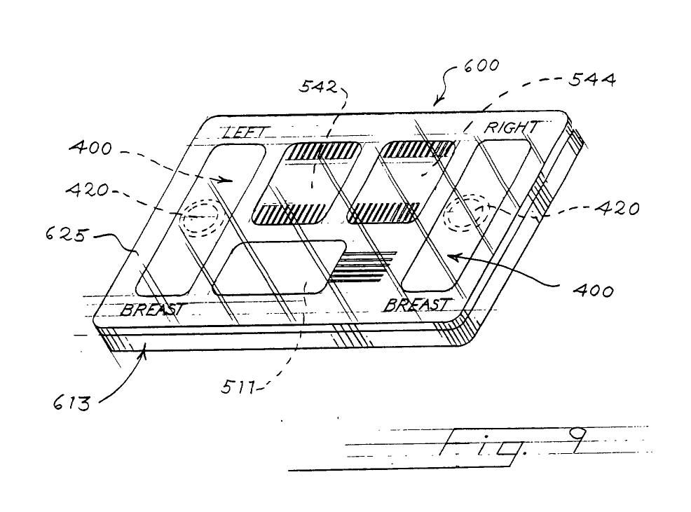

A kit embodiment of the present invention is shown in FIG. 8. Kit 500 includes collection device 400, illustrated in greater detail in FIGS. 6 and 7 hereinabove, together with an exfoliating implement 511 and wipe packets 542 and 544. Wipe packet 542 contains an alcohol wipe or wipes to be used after exfoliation but prior to exudate collection. Wipe packet 544 contains a dry wipe or wipes to be used before or after an exudate aliquant has been collected.

FIG. 9 illustrates another kit embodiment of the present invention. Kit 600 comprises a base 613 provided with appropriate sockets that receive a pair of collection devices 400, due for each breast of the patient, as well as optional exfoliating implement 511 and wipe packets 542 and 544. Transparent removable cover 625 for base 613 preferably is a magnifier such as a Fresnel lens that can be utilized to inspect the respective absorbent pads 420 after an aliquot of a breast exudate has been collected thereon.

Use of the collection devices embodying the present invention is explained below with reference to FIGS. 1 to 3. The protective cover 126 is removed from the opening aperture 116 defined by rim 118. The release strip 124 is also removed to expose the pressure sensitive adhesive 122. The collection device 100 is adhered to the breast such that the aperture 116 circumscribes the nipple by placing rim 118 about the nipple. The position of absorbent pad 120 is then adjusted so as to contact the nipple by raising or lowering the plunger portion 132. The breast is then massaged to express any fluid out of the mammary ducts for collection on the absorbent pad 122. The collection device 100 is then removed from the breast and the collected fluid viewed and/or analyzed.

The maximize the potential for obtaining a mammary fluid sample, and to minimize the chances of contaminating the sample, the nipple region should be first exfoliated with the brush 511 or other exfoliating implement. The nipple area should also be cleaned, preferably with an alcohol wipe. This can be done by wiping the nipple area with an alcohol wipe from packet 542.

The sample collection devices embodying the present invention can be utilized in conjunction with a mammographic examination. More particularly, exudates expressed as a result of mammographic clamping prior to a mammographic examination can be collected and assayed. To that end, the present sample collection device is applied over the nipple of a human breast shortly before mammographic clamping so that any exudate that is expressed as a result of manipulation of the breast during clamping can be captured and retained on the absorbent pad of the sample collection device. Radio-opaque markers on the sample collection device can assist in identifying nipple orientation after clamping.

Patients that secrete a brown or bloody discharge during the mammography procedure can be referred to a physician for further microendoscopic examination and/or lavaged for additional exudate samples which, in turn, can be subjected to a cytological analysis for cell atypia.

The foregoing description is to be taken as illustrative, but not limiting. Still other variants within the spirit and scope of the present invention will readily present themselves to those skilled in the art.

We claim:

1. A sample collection device suitable for collection of an exudate from a mammary duct that terminates at a human breast nipple and comprising:
a hollow receptacle defining an interior volume, and having an open aperture sized to circumscribe the nipple and defined by a rim;
an absorbent pad in the interior volume and positionable to contact the nipple when the receptacle is situated over the nipple; and
a pressure sensitive adhesive on the device for retaining the device on the nipple.

2. The device of claim 1, further comprising a removable cover over the aperture.

3. The device of claim 1, wherein the rim is a flange.

4. The device of claim 3, wherein the flange is a rigid material.

5. The device of claim 3, wherein the flange is a flexible material.

6. The device of claim 1, wherein the absorbent pad in the interior volume is positioned with a reciprocating mechanism to contact the nipple when the receptacle is situated over the nipple.

7. The device of claim 6, wherein the reciprocating mechanism is a plunger portion slidably received in a hollow tubular portion of the receptacle, and wherein the absorbent pad is situated on a distal end of the plunger portion.

8. The device of claim 7 further comprising an observation window at a proximal end of the plunger portion.

9. The device of claim 8 wherein a magnifying lens is mounted to the window.

10. The device of claim 6, wherein the reciprocating mechanism is a flexible hinge between the hollow receptacle and the rim.

11. The device of claim 6, wherein the hollow receptacle has a pleated outside wall portion that is collapsible and extendable so as to position the absorbent pad.

12. The device of claim 11 further comprising an observation window at a proximal end of the hollow receptacle.

13. The device of claim 12 wherein a magnifying lens is mounted to the window.

14. The device of claim 6, wherein the reciprocating mechanism is a flexible hinge region unitary with the hollow receptacle and the rim.

15. The device of claim 14 further comprising an observation window at a proximal end of the hollow receptacle.

16. The device of claim 15 wherein a magnifying lens is mounted to the window.

17. A sample collection device suitable for collection of an exudate from a mammary duct that terminates at a human breast nipple and comprising:
a base sheet;
a pressure sensitive adhesive on the base sheet;
a ring secured to the base sheet and defining a hollow receptacle with an interior volume and terminating in a rim that defines an open aperture sized to circumscribe the nipple;
an absorbent pad situated within the hollow receptacle; and
a sheet-form release material occluding the open aperture and covering exposed adhesive.

18. The sample collection device in accordance with claim 17 wherein the absorbent pad contains a blood highlighting dye.

19. The sample collection device in accordance with claim 18 wherein the dye is hemoccult dye.

20. The sample collection device in accordance with claim 17 wherein the ring includes a radio-opaque marker.

21. The sample collection device in accordance with claim 17 wherein the absorbent pad includes a radio-opaque marker.

22. The sample collection device in accordance with claim 17 wherein the absorbent pad contains a diagnostic composition.

23. A kit for collecting a sample of an exudate from a mammary duct that terminates at a human breast nipple, the kit comprising:
at least one sample collection device suitable for collection of the exudate comprising, a hollow receptacle defining an interior volume and having an open aperture sized to circumscribe the nipple and defined by a rim, an absorbent pad in the interior volume and positionable to contact the nipple when the receptacle is situated over the nipple, and a pressure sensitive adhesive on the device for retaining the device on the nipple;
an exfoliating implement; and
a wipe.

24. The kit of claim 23, wherein the exfoliating implement is a brush.

25. The kit of claim 23, wherein the exfoliating implement is a textured pad.

26. The kit of claim 23, wherein the wipe is an alcohol wipe.

27. The kit of claim 23, wherein the wipe is a dry wipe.

28. A kit for collecting a sample of an exudate from a mammary duct that terminates at a human breast nipple, the kit comprising:
a base;
a pair of sample collection devices on the base and suitable for collection of the exudate, each said device comprising a hollow receptacle defining an interior volume and having an open aperture sized to circumscribed the nipple and defined by a rim, an absorbent pad situated in the interior volume and positionable to contact the nipple when the receptacle is positioned over the nipple, and a pressure sensitive adhesive on each said device for retaining the device on the nipple; and
a transparent cover for the base.

29. The kit in accordance with claim 28 wherein the transparent cover is a magnifier.

30. A method for collecting a sample of mammary duct fluid from a nipple of a human breast through use of a collection device and comprising:
providing at least one sample collection device suitable for collection of the exudate, the collection device comprising a hollow receptacle defining an interior volume and an open aperture sized to circumscribe the nipple and defined by a rim, an absorbent pad in the interior volume and positionable to contact the nipple when the receptacle is situated over the nipple, and a pressure sensitive adhesive on the device;
exfoliating the nipple;
cleaning the nipple;
securing the collection device with the pressure sensitive adhesive on the rim such that the receptacle is situated over the nipple;
positioning the absorbent pad to contact the nipple;
manipulating the breast so as to express an aliquot of any mammary duct fluid present; and
collecting expressed mammary duct fluid on the absorbent pad.

31. The method in accordance with claim 30 wherein the aliquot of mammary duct fluid is collected in conjunction with a mammographic examination.
